(19) United States
(12) Patent Application Publication
(10) Pub. No.: US 2024/0150305 A1
(43) Pub. Date: May 9, 2024

(54) (METH)ACRYLATES CONTAINING A 4-THIAZOLIDINONE UNIT AND POLYMERS THEREOF

(71) Applicant: COMPAGNIE GENERALE DES ETABLISSEMENTS MICHELIN, CLERMONT-FERRAND (FR)

(72) Inventors: Milan FEDURCO, CLERMONT-FERRAND Cedex 9 (FR); Marco RIBEZZO, CLERMONT-FERRAND Cedex 9 (FR)

(21) Appl. No.: 18/548,356

(22) PCT Filed: Feb. 21, 2022
(86) PCT No.: PCT/FR2022/050303

§ 371 Date: Aug. 30, 2023

(30) Foreign Application Priority Data
Mar. 2, 2021 (FR) FR2102009

(51) Int. Cl. C07D 277/04; C07D 415/00; C08F 128/06

(52) U.S. Cl. CPC C07D 277/04; C07D 415/00; C08F 128/06

(57) ABSTRACT

Monomers which contain a 4-thiazolidinone unit linked to a benzene nucleus and which contain a (meth)acrylate function are provided. A process for the synthesis of the monomers and the use thereof in the synthesis of polymers by the radical route is also provided.

This U.S. patent application is a national phase entry of international patent application no. PCT/FR2022/050303, filed Feb. 21, 2022, which claims priority to French patent application no. FR2102009, filed Mar. 2, 2021, the entire contents of which are incorporated herein by reference in their entirety.

### BACKGROUND

### 1. Technical Field

The field of the present invention is that of acrylate or methacrylate monomers comprising a 4-thiazolidinone unit, which are intended for use in the synthesis of polymers by radical polymerization, in particular activated by photoactivatable catalysts.

### 2. Related Art

The synthesis of polymers comprising 4-thiazolidinone units is described in the journal Polymer Chemistry, 2014, 5, 2695. It is carried out by polycondensation reaction between 2-mercaptopropionic acid, a diamine such as hexamethylenediamine and a dialdehyde such as terephthalaldehyde. The authors of the publication highlight that the polycondensate synthesis process is environmentally friendly, because the polycondensation reaction is carried out at ambient temperature in the absence of a catalyst and generates as a by-product only some water.

The formation of water in the polymer inevitably results in the presence of moisture in the polymer. In the case of the use of the polymer in contact with an oxidizable metallic element, it is then necessary to eliminate as much as possible the residual moisture in the polymer to prevent corrosion of the metallic element.

Furthermore, the synthesis of the polymer proves to be very bothersome for the surrounding area, since it is accompanied by a very unpleasant odour due to the use in large amounts of 2-mercaptopropionic acid.

### SUMMARY

The inventors have developed new monomers which make it possible to remedy these drawbacks in the synthesis of polymers containing 4-thiazolidinone units, by using compounds, amine or aldehyde, which are heterodifunctional. Thanks to these new monomers, there is no release of water and no 2-mercaptopropionic acid is used in the synthesis of such polymers containing 4-thiazolidinone units.

Moreover, it is known practice to copolymerize polyurethanes functionalized at the end of the (meth)acrylate chain with conventional (meth)acrylate comonomers, by the radical process. The interest for these prepolymers is growing with the use of 3D printing, because they are polymerizable by photochemical activation. The polymers thus obtained find very varied fields of application ranging from hearing aids to tyres.

Thus, a first subject of the invention is a monomer which contains a 4-thiazolidinone unit linked to a benzene nucleus and which contains a group comprising a (meth)acrylate function, which monomer is of formula (I-1)

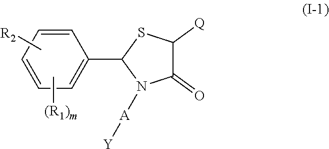

in which Y is (meth)acrylate, Q is methyl, A is a spacer that connects the nitrogen atom of the thiazolidinone ring to Y, R₁ is alkyl, m is an integer ranging from 0 to 4, R₂ is hydrogen or is a group of formula (I-R₂),

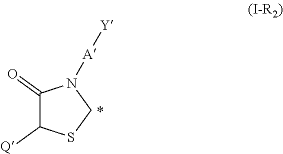

Y′ being identical to Y, Q′ being identical to Q, A′ being identical to A, A′ connecting the nitrogen atom of the thiazolidinone ring to Y′, the symbol * denoting the carbon atom linked to a carbon atom of the benzene nucleus by a covalent bond,

the spacers being a divalent alkanediyl chain which can be interrupted by one or more oxygen atoms or by one or more COO or OCO functions or alternatively be substituted by one or more OH functions.

Another subject of the invention is a process for the synthesis of a monomer in accordance with the invention which comprises two reactions, the first reaction being a condensation reaction of an aldehyde of formula (II), of a compound (III) and of a compound of formula (IV), the second reaction being an esterification reaction to insert the (meth)acrylate function into the reaction product of the first reaction,

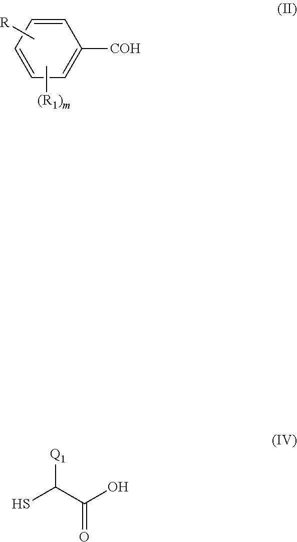

the compound (III) being of formula Y₁-B-NH₂,

Y₁ being hydroxyl or carboxyl, Q₁ being methyl, B being a divalent carbon group which connects the nitrogen atom to Y₁, R₁ being alkyl, m being an integer ranging from 0 to 4, R being hydrogen or an aldehyde function.

The invention also relates to a polymer containing units of a monomer in accordance with the invention.

The invention also relates to a polymer obtained by radical polymerization of a monomer in accordance with the invention, alone or as a mixture with another (meth)acrylate monomer.

The invention also relates to a process for the synthesis of a polymer which comprises the radical polymerization of a monomer in accordance with the invention in the presence of a catalyst which can be photoactivated by irradiation with ultraviolet-visible light.

### DETAILED DESCRIPTION OF EXAMPLE EMBODIMENTS

The compounds mentioned in the description may be of fossil origin or may be biobased. In the latter case, they may be partially or completely derived from biomass or may be obtained from renewable starting materials derived from biomass. Similarly, the compounds mentioned may also originate from the recycling of already-used materials, i.e. they may be partially or totally derived from a recycling process, or obtained from starting materials which are themselves derived from a recycling process.

In the present application and in a known manner, the term hydroxyl means the OH group, and “carboxyl” means the COOH function.

In the present application, when a symbol represents a hydrogen atom, it is said in the present application that it is hydrogen.

The term (meth)acrylate means the acrylate function of formula CH₂═CCH₃—COO— or the methacrylate function of formula CH₂═CCH₃—COO—.

The monomer in accordance with the invention satisfies the formula (I-1)

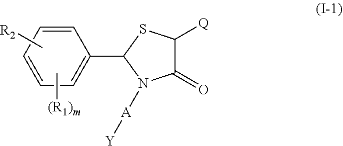

in which Y is (meth)acrylate, Q is methyl, A is a spacer that connects the nitrogen atom of the thiazolidinone ring to Y, R₁ is alkyl, m is an integer ranging from 0 to 4, R₂ is hydrogen or is a group of formula (I-R₂),

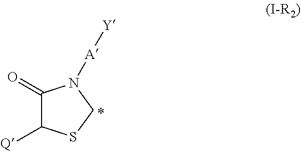

in which Y′ is identical to Y, Q′ is identical to Q, A′ which connects the nitrogen atom of the thiazolidinone ring to Y′ is identical to A, the symbol * denoting the carbon atom linked to a carbon atom of the benzene nucleus by a covalent bond,
the spacers being a divalent alkanediyl chain which can be interrupted by one or more oxygen atoms or by one or more COO or OCO functions or alternatively be substituted by one or more OH functions.

The monomer in accordance with the invention is represented by the following formulae (I-1a) and (I-1b) in which Y is (meth)acrylate, Q is methyl, A is a spacer which connects the nitrogen atom of the thiazolidinone ring to Y; R₁ is alkyl, m is an integer ranging from 0 to 4, R₂ is hydrogen, Y′ is identical to Y; Q′ is identical to Q, A′ which connects the nitrogen atom of the thiazolidinone ring to Y′ is identical to A.

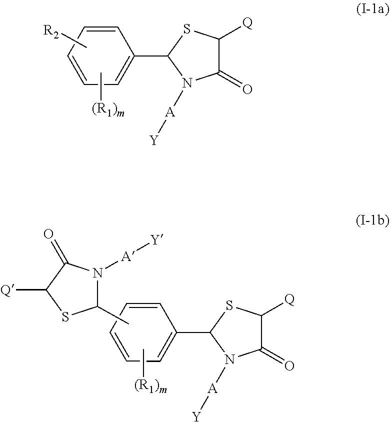

The monomer in accordance with the invention therefore has the essential characteristic of containing a 4-thiazolidinone unit which is directly linked to a substituted or unsubstituted benzene nucleus. It also has the other characteristic of being a compound which contains an acrylate or methacrylate function borne by one or more of the symbols Y, Y′.

In formula (I-1), m is preferentially equal to 0.

When R₂ is different from a hydrogen atom, R₂ is preferentially para or meta relative to the thiazolidinone ring, more preferentially para relative to the thiazolidinone ring.

Within the molecule of formula (I-1), the spacers represented respectively by the symbols A and A′ make it possible to connect the thiazolidinone rings to the functional groups represented respectively by the symbols Y and Y′.

The alkanediyls represented respectively by the symbols A and A′ are preferentially linear. The alkanediyls of the spacers represented respectively by the symbols A and A′ preferentially contain 2 to 20 carbon atoms, more preferentially are ethanediyl.

The alkyl groups in the formulae (I-1), (IR₂) can be linear, branched or cyclic. They are preferentially linear. They preferentially contain 1 to 12 carbon atoms.

The alkyl groups represented by the symbols R₁, R₂ are preferentially alkyls having 1 to 3 carbon atoms, more preferentially are methyl. It follows that the alkyl group represented by the symbol R of formula (II) of the compound useful for the synthesis of the monomer is also preferentially an alkyl having 1 to 6 carbon atoms, more preferentially is methyl.

When m is equal to 0, the monomer of formula (I-1) can be represented by formula (I-2a) or formula (I-2b) in which Y is (meth)acrylate, Q is methyl, A is a spacer which connects the nitrogen atom of the thiazolidinone ring to Y, R₂ is hydrogen, Y′ is identical to Y; Q′ is identical to Q, A′ which connects the nitrogen atom of the thiazolidinone ring to Y′ is identical to A.

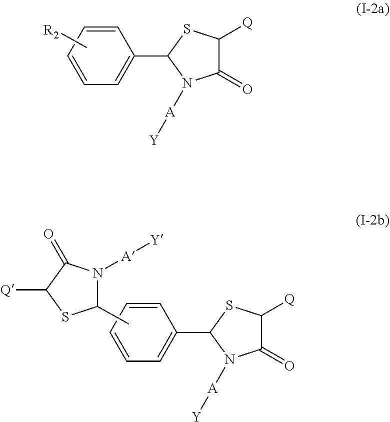

According to the invention, the monomer is of formula (I-3a-acryl), (I-3a-methacryl), (I-3b-acryl) or (I-3b-methacryl), Q and Q′ being methyl.

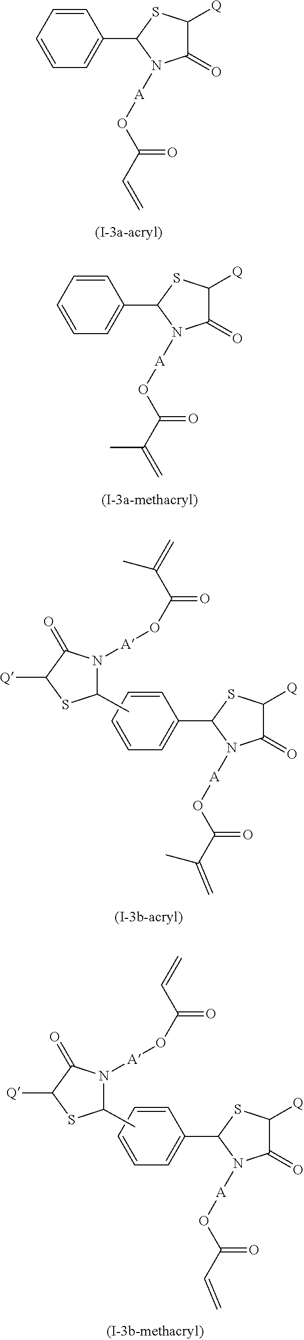

According to a first embodiment of the invention, A and A′ are alkanediyl, preferentially alkanediyls having 2 to 20 carbon atoms in the formulae (I-1a), (1-1b), (I-2a), (I-2b), (I-3a-acryl), (I-3a-methacryl), (I-3b-acryl), (I-3b-methacryl, more preferentially ethanediyl.

According to a second embodiment of the invention, A and A′ have the formula —(C_{y}H_{2y})—CH₂—CH(OH)—CH₂— or —(C_{y}H_{2y})—COO—CH₂—CH₂— in the formulae (I-1a), (I-1b), (I-2a), (I-2b), (I-3a-acryl), (I-3a-methacryl), (I-3b-acryl), (I-3b-methacryl), preferably A and A′ have the formula —(C_{y}H_{2y})—CH₂—CH(OH)—CH₂—. In the formula —(C_{y}H_{2y})—CH₂—CH(OH)—CH₂— and the formula —(C_{y}H_{2y})—COO—CH₂—CH₂—, y integer preferentially varies from 2 to 6. Advantageously, y is equal to 2 due to the commercial availability of the precursors useful for the synthesis of the monomer.

According to a third embodiment of the invention, A and A′ have the formula —(C_{y}H_{2y})—(C_{z}H_{2z}O)ₚ—, y, z and p being integers in the formulae (I-1a), (I-1b), (I-2a), (I-2b), (I-3a-acryl), (I-3a- methacryl), (I-3b-acryl), (I-3b-methacryl). In the formula —(C_{y}H_{2y})—(C_{z}H_{2z}O)ₚ—, y integer preferentially varies from 2 to 6, z preferentially varies from 2 to 6, p preferentially varies from 1 to 20. The values of y and z are advantageously chosen equal to 2 due to the commercial availability of the precursors useful for the synthesis of the monomer.

According to a fourth embodiment of the invention, in the formulae (I-1a), (I-1b), (I-2a), (I-2b), (I-3a-acryl), (I-3a -methacryl), (I-3b-acryl), (I-3b-methacryl), A and A′ are a chain formed from an alkanediyl —(C_{y}H_{2y})— and a polar group of formula CH₂—CH(OH)—CH₂— or —(C_{y}H_{2y})—COO—CH₂—CH₂— interconnected via a polyether chain of formula —(C_{z}H_{2z}O)ₚ—, y, z and p being integers. Preferably, y varies from 2 to 6, z from 2 to 6, p from 1 to 20. The values of y and z are advantageously chosen equal to 2 due to the commercial availability of the precursors useful for the synthesis of the monomer.

As monomers satisfying formula (I-1), mention may be made of the compounds (1) to (5). Mention may also be made of the acrylate homologues thereof.

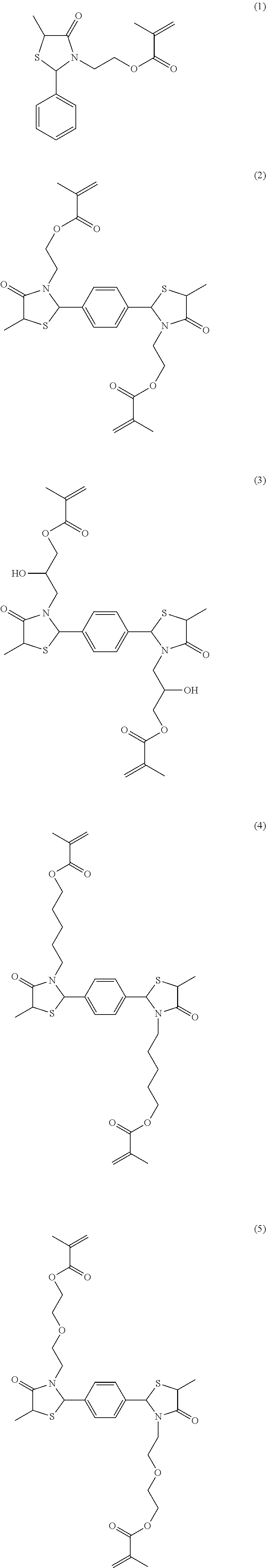 [SMILES: C=C(C)C(=O)OCCN1C(=O)C(C)SC1c1ccccc1 ; C=C(C)C(=O)OCCN1C(=O)C(C)SC1c1ccc(C2SC(C)C(=O)N2CCOC(=O)C(=C)C)cc1 ; C=C(C)C(=O)OCC(O)CN1C(=O)C(C)SC1c1ccc(C2SC(C)C(=O)N2CC(O)COC(=O)C(=C)C)cc1 ; C=C(C)C(=O)OCCCCCN1C(=O)C(C)SC1c1ccc(C2SC(C)C(=O)N2CCCCCOC(=O)C(=C)C)cc1 ; C=C(C)C(=O)OCCOCCN1C(=O)C(C)SC1c1ccc(C2SC(C)C(=O)N2CCOCCOC(=O)C(=C)C)cc1]

The monomer in accordance with the invention is typically prepared by a process which comprises two reactions.

The first reaction is a condensation reaction of an aldehyde of formula (II), of a compound (III) and of a compound of formula (IV) to form the intermediate compounds of formula (V),

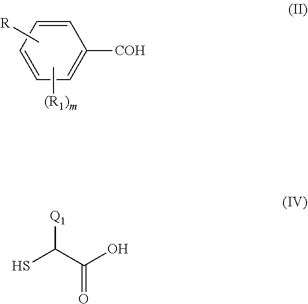

the compound (III) being of formula Y₁-B-NH₂, Y₁ being hydroxyl or carboxyl, Q₁ being methyl, B being a divalent group which connects the nitrogen atom to Y₁, R₁ being alkyl, m being an integer ranging from 0 to 4, R being hydrogen or an aldehyde function.

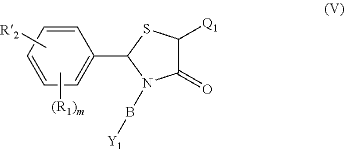

in which Y₁ is hydroxyl or carboxyl, Q₁ is methyl, B is a divalent carbon group which connects the nitrogen atom of the thiazolidinone ring to Y₁, R₁ is alkyl, m is an integer ranging from 0 to 4, R′₂ is hydrogen, or a group of formula (V-R₂),

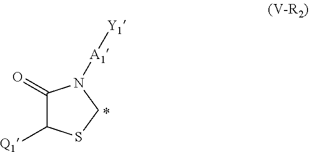

Y₁′ being hydroxyl or carboxyl, Q₁′ being methyl, A₁′ being a divalent carbon group which connects the nitrogen atom of the thiazolidinone ring to Y₁′, the symbol * denoting the carbon atom linked to a carbon atom of the benzene nucleus by a covalent bond, Y₁′ being identical to Y₁, A₁′ being identical to A₁, Q₁′ being identical to Q₁.

The first reaction leads to the synthesis of the intermediate compound of formula (V) according to the reaction scheme in two steps A and B below.

When R represents an aldehyde function, the first reaction of the process leads to an intermediate compound of formula (V) in which R has the formula (V-R₂), the second reaction leads to the monomer of formula (I-1) in which R₂ has the formula (I-R₂).

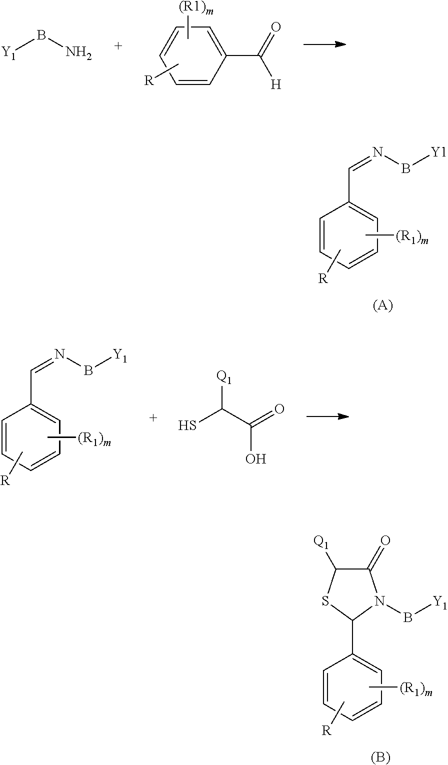

The second reaction is a reaction to insert the (meth)acrylate function into the intermediate compound of formula (V).

When the intermediate compound of formula (V) is an alcohol, the second reaction is typically an esterification reaction of the alcohol with (meth)acrylic acid. Alternatively, instead of (meth)acrylic acid, the acid chloride of (meth)acrylic acid can be used.

When the intermediate compound of formula (V) is an acid, the second reaction is typically an esterification reaction of an alcohol bearing a (meth)acrylate function with the acid. The alcohol bearing a (meth)acrylate function is, for example, 2-hydroxyethyl (meth)acrylate.

For example, monomers according to the invention can be prepared by a process involving one or other of the following reaction sequences:

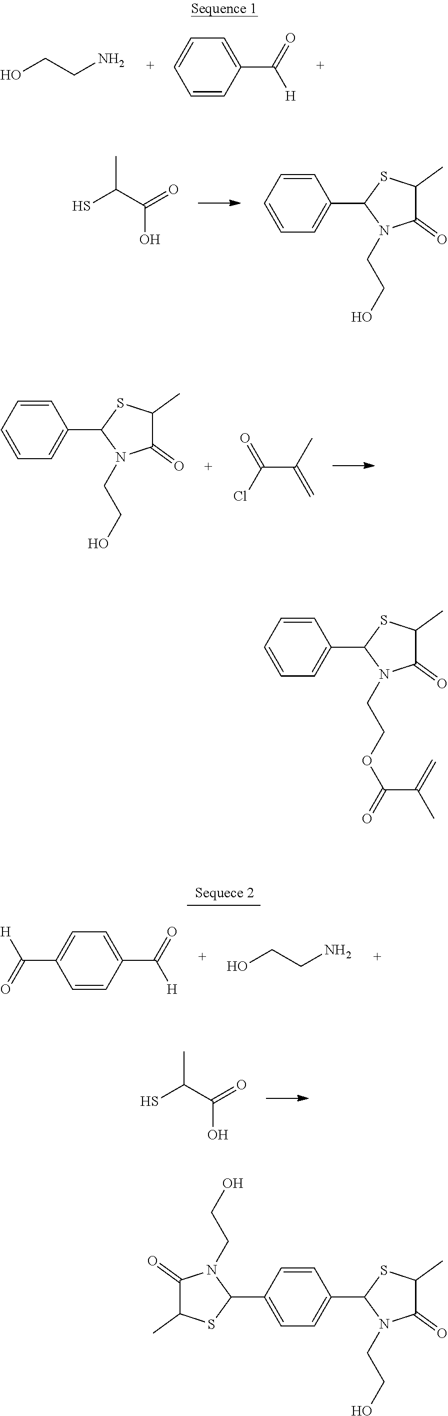

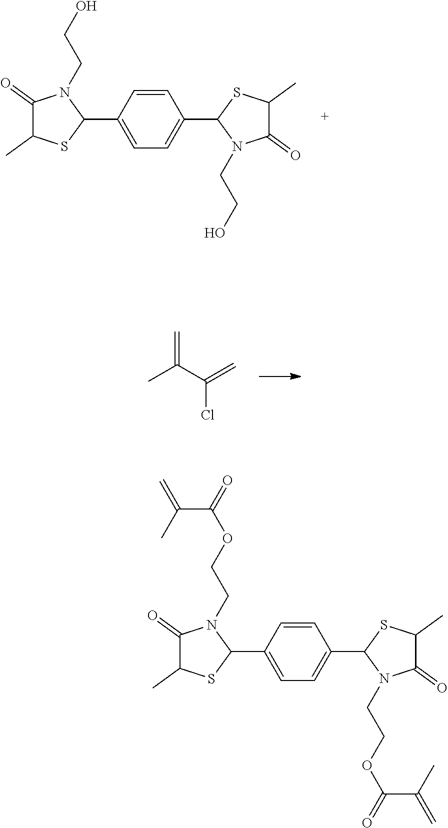

The reaction conditions for synthesizing the monomer are adapted by those skilled in the art according to the reaction scheme involved and taking into account the solubility of each of the substrates and the stoichiometry of the reaction.

The monomers in accordance with the invention are used in processes for the synthesis of polymers by polymerization reaction of the (meth)acrylate function, in particular in the presence of molecules which are activated when they are exposed to irradiation with ultraviolet-visible light. Such molecules are photoactivatable catalysts of which the role is to activate the initiation reaction for the radical polymerization reaction of the (meth)acrylate function.

As a photoactivatable catalyst, mention may be made of 2,4,6-trimethylbenzoylphenyl phosphinate sold under the name “Irgacure TPO-L”, phenylbis(2,4,6-trimethylbenzoyl)phosphine oxide sold under the name “Irgacure 819”, diphenyl(2,4,6-trimethylbenzoyl)phosphine oxide sold under the name “Irgacure TPO”, 2-hydroxy-4′-(2-hydroxyethoxy)-2-methylpropiophenone sold under the name “Irgacure 2959”.

The monomer in accordance with the invention can be polymerized alone or be copolymerized with another monomer, in particular (meth)acrylate. As comonomer, mention may be made of a monomer which is also in accordance with the invention and which differs from the first by its chemical structure. As comonomer, any monomer polymerizable by the radical route may be suitable. As such, mention may be made, for example, of monomers comprising an acrylate or methacrylate function, such as isobornyl acrylate, lauryl methacrylate, diethylene glycol monomethyl ether methacrylate, hexanediol diacrylate, di(ethylene glycol) diacrylate, ethylene glycol dimethacrylate, trimethylolpropane trimethacrylate.

The monomer in accordance with the invention can also be copolymerized with a prepolymer bearing functions which can be polymerized by the radical route, in particular (meth)acrylates. By way of prepolymers, mention may very particularly be made of polyurethanes bearing a (meth)acrylate function at their chain ends, such as the photoactivatable polyurethane prepolymers sold for example under the name “Trixene” (Chemtura/Lanxess, Italy), “Bomar” (DYMAX, USA), or “Ebecryl” (Allnex, USA). The incorporation of monomer units according to the invention in such polyurethane prepolymers functionalized at the end of the (meth)acrylate chain leads to an improvement in the elongation at break compared to the incorporation of conventional (meth)acrylate monomer units lacking a 4-thiazolidinone unit.

The monomers in accordance with the invention also have the advantage of polymerizing in a few minutes, typically in less than 3 minutes, under irradiation with ultraviolet-visible light in the presence of a photoactivatable catalyst.

The monomers in accordance with the invention therefore show all their advantage in the synthesis of polymers by 3D printing under irradiation with ultraviolet light.

In summary, the invention is advantageously performed according to any one of the following Embodiments 1 to 17:

Embodiment 1: Monomer which contains a 4-thiazolidinone unit linked to a benzene nucleus and which contains a group comprising a (meth)acrylate function, which monomer has the formula (I-1)

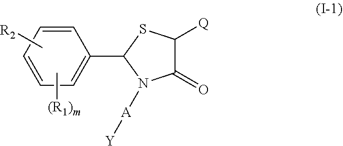

in which Y is (meth)acrylate, Q is methyl, A is a spacer that connects the nitrogen atom of the thiazolidinone ring to Y, R₁ is alkyl, m is an integer ranging from 0 to 4, R₂ is hydrogen or is a group of formula (I-R₂),

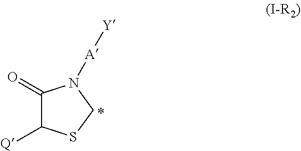

Y′ being identical to Y, Q′ being identical to Q, A′ being identical to A, A′ connecting the nitrogen atom of the thiazolidinone ring to Y′, the symbol * denoting the carbon atom linked to a carbon atom of the benzene nucleus by a covalent bond,

the spacers being a divalent alkanediyl chain which can be interrupted by one or more oxygen atoms or by one or more COO or OCO functions or alternatively be substituted by one or more OH functions.

Embodiment 2: Monomer according to Embodiment 1, in which R₂ is para or meta to the thiazolidinone ring when it is different from a hydrogen atom.

Embodiment 3: Monomer according to Embodiment 1 or 2, in which R₂ is para to the thiazolidinone ring when it is different from a hydrogen atom.

Embodiment 4: Monomer according to any one of Embodiments 1 to 3, in which m is equal to 0.

Embodiment 5: Monomer according to any one of Embodiments 1 to 4, in which the alkyls represented by R₁, R₂ are alkyls having 1 to 3 carbon atoms.

Embodiment 6: Monomer according to any one of Embodiments 1 to 5, in which the alkyls represented by R₁, R₂, are methyl.

Embodiment 7: Monomer according to any one of Embodiments 1 to 6, in which A and A′ are alkanediyl.

Embodiment 8: Monomer according to any one of Embodiments 1 to 7, in which A and A′ are alkanediyls having 2 to 20 carbon atoms.

Embodiment 9: Monomer according to any one of Embodiments 1 to 8, in which A and A′ are ethanediyl.

Embodiment 10: Monomer according to any one of Embodiments 1 to 6, in which A and A′ have the formula —(C_{y}H_{2y})—CH₂—CH(OH)—CH₂— or —(C_{y}H_{2y})—COO—CH₂—CH₂—, y being an integer.

Embodiment 11: Monomer according to any one of Embodiments 1 to 6, in which A and A′ have the formula —(C_{y}H_{2y})—(C_{z}H_{2z}O)ₚ—, y, z and p being integers.

Embodiment 12: Monomer according to any one of Embodiments 1 to 6, in which A and A′ are a chain formed from an alkanediyl —(C_{y}H_{2y})— and a polar group of formula CH₂—CH(OH)—CH₂— or —(C_{y}H_{2y})—COO—CH₂—CH₂— interconnected via a polyether chain of formula —(C_{z}H_{2z}O)ₚ—, y, z and p being integers.

Embodiment 13: Monomer according to any one of Embodiments 10 to 12, in which y is equal to 2.

Embodiment 14: Process for the synthesis of a monomer defined in any one of Embodiments 1 to 13, which comprises two reactions, the first reaction being a condensation reaction of an aldehyde of formula (II), of a compound (III) and of a compound of formula (IV), the second reaction being an esterification reaction to insert the (meth)acrylate function into the reaction product of the first reaction,

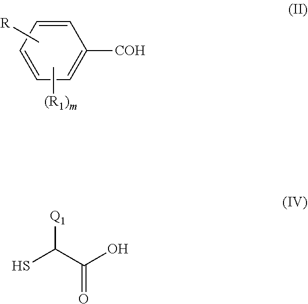

the compound (III) being of formula Y₁-B-NH₂,

Y₁ being hydroxyl or carboxyl, Q₁ being methyl, B being a divalent carbon group which connects the nitrogen atom to Y₁, R₁ being alkyl, m being an integer ranging from 0 to 4, R being hydrogen or an aldehyde function.

Embodiment 15: Polymer containing units of a monomer defined in any one of Embodiments 1 to 13.

Embodiment 16: Polymer obtained by radical polymerization of a monomer defined in any one of Embodiments 1 to 13, alone or in a mixture with another (meth)acrylate monomer.

Embodiment 17: Process for the synthesis of a polymer which comprises the radical polymerization of a monomer defined in any one of Embodiments 1 to 13 in the presence of a catalyst photoactivatable by irradiation with ultraviolet-visible light.

The abovementioned characteristics of the present invention, and also others, will be better understood on reading the following description of several exemplary embodiments of the invention, which are given as nonlimiting illustrations.

### EXAMPLES

Thermal characterizations by differential scanning calorimetry (DSC) were carried out with a device Mettler Toledo DSC3. The sample (about 10 mg) is weighed and sealed in a 40 μl aluminium crucible. The crucible is pierced with a fine needle just before the measurement. Dry nitrogen is used as purge gas with a flow of 30 ml min⁻¹. Two successive cycles of heating and cooling are carried out identically. For each cycle, the sample and also an empty reference crucible are heated from a minimum temperature to a maximum temperature at 10° C. min⁻¹. The temperature is maintained for 2 minutes at the maximum temperature, then cooled from the maximum temperature to the minimum temperature at 10° C. min⁻¹. Unless otherwise indicated, the glass transition temperature (Tg) and the melting temperature (Tm) are measured in the second cycle. If appropriate, in the event of mass loss observed during the second cycle, a third cycle is carried out under the same conditions as the second cycle. The glass transition temperature corresponds to the temperature which is the midpoint temperature (Tmg) as defined in Standard ASTM D3418-99. The melting temperature (Tm) corresponds to the tip of the melting peak.

All of the syntheses are carried out under a nitrogen atmosphere, unless otherwise indicated.

### Example 1 According to the Invention

The 2-(5-methyl-4-oxo-2-phenylthiazolidin-3-yl)ethyl methacrylate monomer is synthesized according to the following reaction sequence with the synthesis of 3-(2-hydroxyethyl)-5-methyl-2-phenylthiazolidin-4-one as intermediate product:

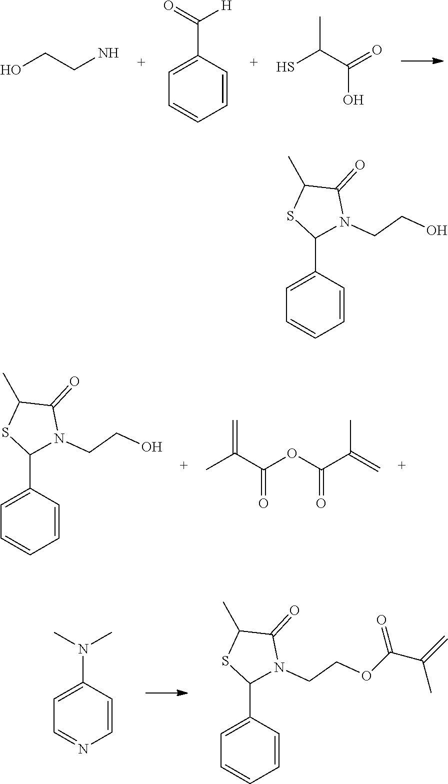

Synthesis of the intermediate product, 3-(2-hydroxyethyl)-5-methyl-2-phenylthiazolidin-4-one, from 2-mercaptopropionic acid, ethanolamine and benzaldehyde:

A 100 ml, four-necked flask, dried beforehand under vacuum at more than 100° C. and equipped with a condenser, bubbler, thermometer, dropping funnel and magnetic stirrer bar, is charged with 21.23 g (i.e. 200 mmol) of 99% benzaldehyde (Merck B1334). 12.22 g (i.e. 200 mmol) of 99% ethanolamine (Sigma Aldrich 411000) are added dropwise and with stirring; the exothermicity is maintained at approximately 60° C. using an ice bath. At the end of the addition of the amine, 22.35 g (i.e. 200 mmol) of 95% 2-mercaptopropionic acid (Sigma Aldrich W318000) are introduced; the exothermicity is always maintained at 60° C. with the ice bath. The reaction medium is stirred at 75° C. for 48 hours under a gentle stream of nitrogen. The viscous liquid obtained is purified by chromatography with the heptane/ethyl acetate/methanol solvent mixture (10:6:4). The product is analysed by nuclear magnetic resonance (NMR) analysis.

1H NMR analysis 600 MHz, DMSO-d6 of the product gave the following results:

1H NMR (600 MHz, DMSO-d6) δ7.46-7.19 (m, 5H), 5.83 (s, 1H), 4.74 (t, J=5.6 Hz, 1H), 4.12-3.88 (m, 1H), 3.55 (dt, J=13.7, 6.0 Hz, 1H), 3.42 (dd, J=11.0, 6.1 Hz, 1H), 3.31-3.23 (m, 1H), 2.59 (dt, J=13.7, 6.2 Hz, 1H), 1.48 (d, J=7.0 Hz, 3H).

### Synthesis of Methacrylate

3-(2-Hydroxyethyl)-5-methyl-2-phenylthiazolidin-4-one (475 mg, 2 mmol) and methacrylic acid anhydride (300 μl, 2 mmol) are dissolved in 70 ml of acetonitrile, then 4.89 mg of dimethylaminopyridine (4.89 mg, 0.04 mmol) are added. The reaction medium is heated at 55° C. for 18 hours. The reaction medium is then filtered through a 5 μm nylon filter. Then 8 mg of inhibitor, 4-methoxyphenol (MEHQ), are added to the solution and the acetonitrile is removed by distillation at 50° C. using a rotary evaporator. A light yellow oil is obtained and is diluted in 30 ml of chloroform. The residual methacrylic anhydride is hydrolysed to sodium methacrylate under basic conditions by adding 10 ml of a NaHCO₃ buffer solution (pH 9) in a separating funnel. The organic phase is recovered. A further 30 ml of CHCl₃ are added for the extraction operation which is repeated a second time. The recovered organic phases are washed with water, then treated with 1% hydrochloric acid until a neutral pH of the aqueous phase is obtained. The organic phase is dried over anhydrous sodium sulfate, and filtered through a 5 μm nylon filter. Then 8 mg MEHQ are added to the organic phase, and the chloroform is removed by distillation to isolate the methacrylate.

### Polymerization of Methacrylate

100 mg of the methacrylate with 2% by weight of Irgacure 819 are placed in a 2 ml flask fitted with a silicone stopper (“snap cap”). The assembly is purged with argon, then exposed under a “Henley” lamp (Uvaprint 100-200 HPCV2 UV lamp (Honle/Germany), wavelength: 200-800 nm) for 3 minutes. A solid is obtained, which is a polymer with a glass transition temperature of 35° C. determined by DSC analysis. No melting is observed in the temperature range extending up to 260° C.

### Example 2 According to the Invention

The 2-hydroxy-3-[2-(5-methyl-4-oxo-2-phenylthiazolidin-3-yl)-ethoxy]propyl methacrylate monomer is synthesized according to the following reaction sequence with the synthesis of 3-(2-hydroxyethyl)-5-methyl-2-phenylthiazolidin-4-one as intermediate product:

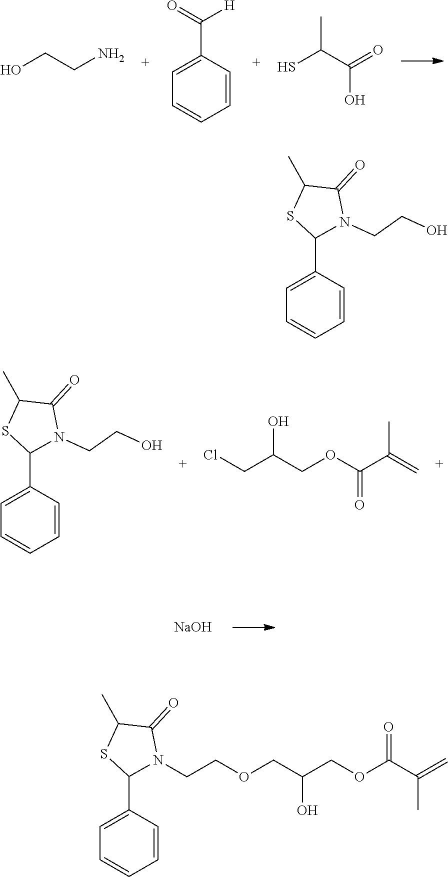

The intermediate product, 3-(2-hydroxyethyl)-5-methyl-2-phenylthiazolidin-4-one, is synthesized according to the procedure described in Example 1.

A 4-necked 100 ml flask previously dried under vacuum at more than 100° C., equipped with a condenser, bubbler, thermometer and magnetic stirrer bar, is charged with 0.16 g of NaOH (i.e. 4 mmol), then 30 ml of ethanol anhydrous under stirring until complete dissolution of the sodium hydroxide. 0.475 g (i.e. 2 mmol) of 3-(2-hydroxyethyl)-5-methyl-2-phenylthiazolidin-4-one is added. Then, 0.357 g (2 mmol) of 3-chloro-2-hydroxypropyl methacrylate is added in six 50 μl aliquots every 10 minutes (NaCl deposit on the walls of the round-bottomed flask). The reaction medium is stirred at the temperature of 23° C. for 16 hours under a gentle stream of nitrogen. Ethanol is removed by distillation at 40° C. Then 50 ml of ethyl acetate are added, followed by 10 ml of 1% hydrochloric acid. The organic phase is separated in a separating funnel and is washed twice with 10 ml of water. Then, 5 mg of an inhibitor, 4-methylphenol (200 ppm), are added and the ethyl acetate is removed by distillation at 40° C. A viscous transparent liquid is obtained. The monomer is purified by chromatography with a mixture of heptane:ethyl acetate and methanol (5:5:1, by volume).

### Example 3 According to the Invention

The monomer is synthesized according to the following reaction sequence with the synthesis of 2,2′-(1,4-phenylene)bis[3-(2-hydroxyethyl)-5-methyl-1,3-thiazolidin-4-one] as intermediate product:

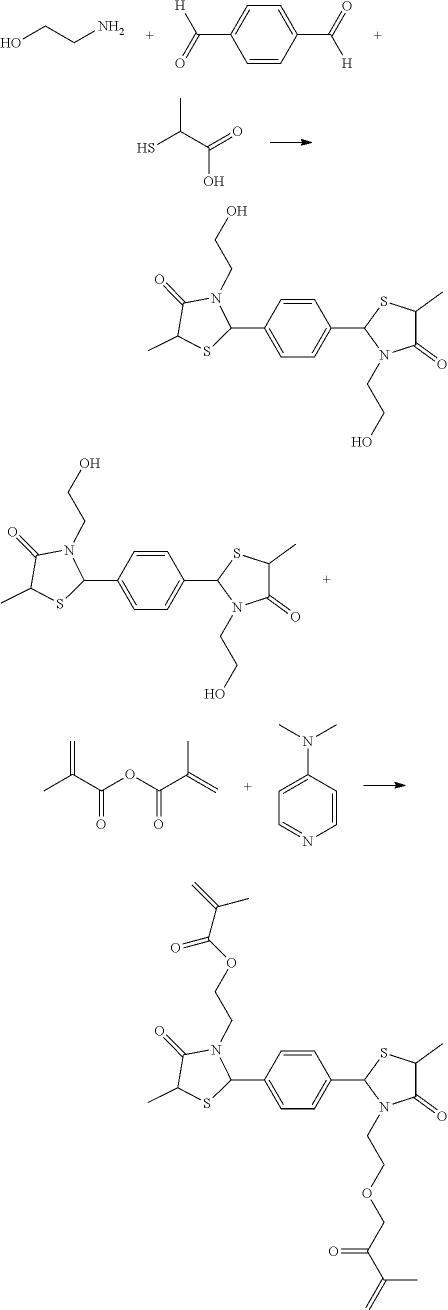

Synthesis of the intermediate product, 2,2′-(1,4-phenylene)bis[3-(2-hydroxyethyl)-5-methyl-1,3-thiazolidin-4-one], from terephthalaldehyde, ethanolamine and 2-mercaptopropionic acid:

A 100 ml, four-necked flask, dried beforehand under vacuum at more than 100° C. and equipped with a condenser, bubbler, thermometer and magnetic stirrer bar, is charged with 13.4 g (i.e. 100 mmol) of terephthalaldehyde followed by 44.69 g (i.e. 400 mmol) of 95% 2-mercaptopropionic acid. An exothermicity to around 40° C. is observed and the mixture is stirred at ambient temperature until the solid dissolves. 12.22 g (i.e. 200 mmol) of ethanolamine are then added in one go; exothermicity is observed to around 105° C. The reaction medium is stirred for 24 hours at ambient temperature under a gentle stream of nitrogen. After 24 hours of reaction, a 10 g aliquot is taken which is dissolved in 300 ml of butanone (ethyl methyl ketone) at 40° C. The organic phase is washed with 3×300 ml of 0.6M NaOH (pH 9) and 3×300 ml of 20% NaCl. The aqueous phase is re-extracted with 3×300 ml of butanone. The combined organic phases are dried over Na₂SO₄, filtered and evaporated. 7 g of a viscous yellow liquid are obtained, this being purified on silica gel using an ethyl acetate and heptane solvent mixture. A white solid is isolated after purification.

H NMR (600 MHz, DMSO) δ7.50-7.27 (m, 2H), 6.00-5.74 (m, 1H), 4.78 (t, J=5.5 Hz, 1H), 4.00 (qd, J=7.1, 6.1 Hz, 1H), 3.58 (dt, J=13.7, 5.7 Hz, 1H), 3.45 (dd, J=11.1, 5.9 Hz, 1H), 3.35 (dd, J=11.0, 5.6 Hz, 1H), 2.60 (dt, J=13.6, 6.4 Hz, 1H), 1.56-1.41 (m, 3H).

### Synthesis of Methacrylate

2,2′-(1,4-phenylene)bis[3-(2-hydroxyethyl)-5-methyl-1,3-thiazolidin-4-one] (793 mg, 2 mmol) and methacrylic acid anhydride (630 μl, 2 mmol) are dissolved in 70 ml of acetonitrile, then 4.89 mg of dimethylaminopyridine (0.04 mmol) are added. The reaction medium is heated at 55° C. for 15 hours. The reaction medium is then filtered through a 5 μm nylon filter. Then 8 mg of inhibitor, the 4-methoxyphenol (MEHQ), are added to the solution and the acetonitrile is removed by distillation at 50° C. using a rotary evaporator. A light yellow oil is obtained and is diluted in 30 ml of chloroform.

The residual methacrylic anhydride is hydrolysed to sodium methacrylate under basic conditions by adding 10 ml of a NaHCO₃ buffer solution (pH 9) in a separating funnel. The organic phase is recovered. A further 30 ml of CHCl₃ are added for the extraction operation which is repeated a second time. The recovered organic phases are washed with water, then treated with 1% hydrochloric acid until a neutral pH of the aqueous phase is obtained. The organic phase is dried over anhydrous sodium sulfate, and filtered through a 5 μm nylon filter. Then 8 mg MEHQ are added to the organic phase, and the chloroform is removed by distillation to isolate the methacrylate.

### Polymerization of Methacrylate

100 mg of the methacrylate with 2% by weight of Irgacure 819 are placed in a 2 ml flask fitted with a silicone stopper (“snap cap”). The assembly is purged with argon, then exposed under a “Henley” lamp (Uvaprint 100-200 HPCV2 UV lamp (Honle/Germany), wavelength: 200-800 nm) for 3 minutes. A solid is obtained, which is a polymer with a glass transition temperature of 154° C. determined by DSC analysis. No melting is observed in the temperature range extending up to 260° C.

1. A monomer which contains a 4-thiazolidinone unit linked to a benzene nucleus and which contains a group comprising a (meth)acrylate function, which monomer has the formula (I-1) 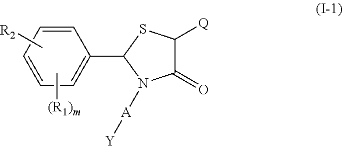
in which Y is (meth)acrylate, Q is methyl, A is a spacer that connects the nitrogen atom of the thiazolidinone ring to Y, R₁ is alkyl, m is an integer ranging from 0 to 4, R₂ is hydrogen or is a group of formula (I-R₂), 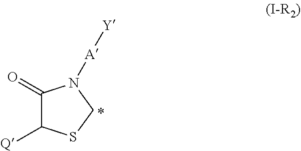
Y′ being identical to Y, Q′ being identical to Q, A′ being identical to A, A′ connecting the nitrogen atom of the thiazolidinone ring to Y′, the symbol * denoting the carbon atom linked to a carbon atom of the benzene nucleus by a covalent bond,
the spacers being a divalent alkanediyl chain which can be interrupted by one or more oxygen atoms or by one or more COO or OCO functions or alternatively be substituted by one or more OH functions.

2. The monomer according to claim 1, in which m is equal to 0.

3. The monomer according to claim 1, in which A and A′ are alkanediyl, preferably ethanediyl.

4. The monomer according to claim 1, in which A and A′ have the formula —(C_{y}H_{2y})—CH₂—CH(OH)—CH₂— or —(C_{y}H_{2y})—COO—CH₂—CH₂—, y being an integer.

5. The monomer according to claim 1, in which A and A′ have the formula —(C_{y}H_{2y})—(C_{z}H_{2z}O)ₚ—, y, z and p being integers.

6. The monomer according to claim 1, in which A and A′ are a chain formed from an alkanediyl —(C_{y}H_{2y})— and a polar group of formula CH₂—CH(OH)—CH₂— or —(C_{y}H_{2y})—COO—CH₂—CH₂— interconnected via a polyether chain of formula —(C_{z}H_{2z}O)ₚ—, y, z and p being integers.

7. A process for the synthesis of a monomer according to any one of claim 1 which comprises two reactions, the first reaction being a condensation reaction of an aldehyde of formula (II), of a compound (III) and of a compound of formula (IV), the second reaction being an esterification reaction to insert the (meth)acrylate function into the reaction product of the first reaction, 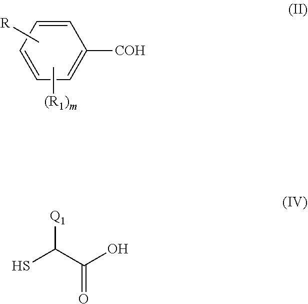
the compound (III) being of formula Y₁-B-NH₂, Y₁ being hydroxyl or carboxyl, Q₁ being methyl, B being a divalent carbon group which connects the nitrogen atom to Y₁, R₁ being alkyl, m being an integer ranging from 0 to 4, R being hydrogen or an aldehyde function, one of the symbols Y₁, Q₁ and R containing a hydroxyl or carboxyl function.

8. A polymer containing units of a monomer defined in any one of claim 1.

9. The polymer obtained by radical polymerization of a monomer defined in claim 1, alone or as a mixture with another (meth)acrylate monomer.

10. A process for the synthesis of a polymer which comprises the radical polymerization of a monomer defined in claim 1 in the presence of a catalyst photoactivatable by irradiation with ultraviolet-visible light.

11. The monomer according to claim 3, in which A and A′ are ethanediyl.
